(19) United States
(12) Patent Application Publication
(10) Pub. No.: US 2018/0296745 A1
(43) Pub. Date: Oct. 18, 2018

(54) DEVICE AND METHOD FOR GENERATING A FILTERED PRESSURE SIGNAL

(71) Applicant: GAMBRO LUNDIA AB, Lund (SE)

(72) Inventors: Bo OLDE, Lund (SE); Kristian SOLEM, Trelleborg (SE)

(21) Appl. No.: 15/569,483

(22) PCT Filed: Jun. 3, 2016
(86) PCT No.: PCT/EP2016/062620

§ 371 Date: Oct. 26, 2017

(30) Foreign Application Priority Data
Jun. 25, 2015 (SE) 1550883-1

(51) Int. Cl. A61M 1/36; G16H 40/63

(52) U.S. Cl. CPC A61M 1/3639; A61M 1/3656; A61M 2230/30; A61M 2205/3331; A61M 2205/50; G16H 40/63

(57) ABSTRACT

A signal filtering device acquires one or more pressure signals that comprise first pulses originating from a first pulse generator associated with a fluid containing system and second pulses. The signal filtering device populates (504) signal vectors by signal segments in the pressure signal(s) and computes (505) principal components for the signal vectors by Principal Component Analysis (PCA). A filtered signal is generated by selecting (506) a first subset of principal components mainly representing the first pulses, determining (507) associated weights, and subtracting (508) the principal components in the first subset from the pressure signal as a function of the weights. Alternatively, a filtered signal is generated by selecting (506) a second subset of principal components that are excluded from the first subset, determining (507) associated weights, and combining (508) the principal components in the second subset as a function of the weights. The signal filtering device may be associated with or included in an apparatus for extracorporeal blood processing, such as a dialysis machine.

### TECHNICAL FIELD

The present invention relates to processing of a pressure signal obtained from a pressure sensor in a fluid containing system, and in particular to filtering of the pressure signal for suppression of pulses originating from a pulse generator in the fluid containing system. The present invention is e.g. applicable in fluid containing systems for extracorporeal blood treatment.

### BACKGROUND ART

In extracorporeal blood processing, blood is taken out of a human subject, processed (e.g. treated) and then reintroduced into the subject by means of an extracorporeal blood flow circuit (“EC circuit”) which is part of a blood processing apparatus. Generally, the blood is circulated through the EC circuit by a blood pump. In certain types of extracorporeal blood processing, the EC circuit includes an access device for blood withdrawal (e.g. an arterial needle or catheter) and an access device for blood reintroduction (e.g. a venous needle or catheter), which are inserted into a dedicated blood vessel access (e.g. fistula or graft) on the subject. This type of EC circuit is, e.g., used in extracorporeal blood treatments such as hemodialysis, hemodiafiltration, hemofiltration, plasmapheresis, bloodbanking, blood fraction separation (e.g. cells) of donor blood, apheresis, extracorporeal blood oxygenation, assisted blood circulation, extracorporeal liver support/dialysis, ultrafiltration, etc.

It is vital to minimize the risk for malfunctions in the EC circuit, since these may lead to a potentially life-threatening condition of the subject. Serious conditions may e.g. arise if the EC circuit is disrupted downstream of the blood pump, e.g. by a Venous Needle Dislodgement (VND) event, in which the venous needle comes loose from the blood vessel access. Such a disruption may cause the subject to be drained of blood within minutes. WO97/10013, US2005/0010118, WO2009/156174, WO2010/149726 and US2010/0234786 all propose various techniques for detecting a VND event by identifying an absence of heart or breathing pulses in a pressure signal from a pressure sensor (“venous pressure sensor”) on the downstream side of the blood pump in the EC circuit.

Recently, it has also been shown to be possible to monitor and analyze the behavior of physiological pressure generators such as the heart or respiratory system, based on pressure recordings in the EC circuit. Various applications are found in WO2010/149726, WO2011/080189, WO2011/080190, WO2011/080191, WO2011/080194 and WO2014/147028 which are incorporated herein by reference. For example, these applications include monitoring a subject's heart pulse rate, blood pressure, heart rhythm, cardiac output, blood flow rate through the blood vessel access (“access flow”), arterial stiffness, as well as identifying signs of stenosis formation within the blood vessel access, predicting rapid symptomatic blood pressure decrease and detecting, tracking and predicting various breathing disorders, and detecting cardiac arrest.

Furthermore, WO2011/080188 proposes a technique for identifying and signaling a reverse placement of the devices for blood withdrawal and blood reintroduction in the vascular access by detecting and analyzing physiological pulses in a pressure signal recorded in the EC circuit.

All of these monitoring techniques presume that the physiological pulses can be reliably detected in the pressure signal. To enable monitoring, it may be necessary to filter the pressure signal for removal or suppression of signal interferences. The signal interferences comprise pressure pulses (“pump pulses”) originating from the blood pump, and may also comprise further interfering pressure pulses, e.g. caused by further pumps, valves, balancing chambers, etc in the EC circuit. It may be a challenging task to properly remove e.g. the pump pulses, since the rate of the physiological pulses and the rate of the blood pump, i.e. the blood flow through the EC circuit, may change over time. If the rate of physiological pulses matches the rate of pump pulses, it is not unlikely that the filtering will remove also the physiological pulses, causing the monitoring technique to fail. Filtering is also rendered difficult by the fact that the pump pulses generally are much stronger than the physiological pulses in the pressure signal. To address these problems, WO2009/156175 proposes that the pressure signal is filtered in the time-domain, by subtraction of a predicted signal profile of the pressure pulses originating from the blood pump. The predicted signal profile may be obtained by reference measurements or by simulations. In one implementation, the predicted signal profile is retrieved from a library of pre-stored reference profiles, based on the current operating frequency of the blood pump, and subtracted from the pressure signal, based on timing information given by a dedicated pump sensor or by a control signal for the blood pump. In another implementation, the predicted signal profile is retrieved and subtracted by a best match technique, in which the predicted signal profile is scaled and shifted so as to minimize differences to the pressure signal before the subtraction. In yet another implementation, the predicted signal profile and the pressure signal are input to an adaptive filter that iterates to generate an error signal which is essentially free of the signal interferences caused by the blood pump.

WO97/10013 proposes a different filtering technique denoted “notch-equivalent filter”, which presumes that the frequency and phase of the blood pump are known. Sinus signals are generated at the known frequency and at multiples of the known frequency. The sinus signals are input to an adaptive filter, which adapts the amplitude and the phase of each sinus signal to the pressure signal to be filtered. The sinus signals are then subtracted from the pressure signal at the respective amplitude and phase.

There is a continued need to achieve an improved filtering technique, in terms of one or more of the following: ability to handle overlap in frequency and/or time between pump pulses and physiological pulses, complexity of the filtering technique, ability to generate the filtered signal in real time, processing efficiency and memory usage during filtering, accuracy of the filtered signal, and robustness of the filtering technique.

Corresponding needs may arise in other fields of technology. Thus, generally speaking, there is a need for an improved technique for filtering a time-dependent pressure signal obtained from a pressure sensor in a fluid containing system so as to essentially remove first pulses originating from a first pulse generator in the fluid containing system while retaining second pulses of other origin.

### SUMMARY

It is an objective of the invention to at least partly overcome one or more limitations of the prior art.

Another objective is to provide a filtering technique capable of meeting one or more of the above-mentioned needs.

One or more of these objectives, as well as further objectives that may appear from the description below, are at least partly achieved by signal filtering devices, a filtering method and a computer-readable medium according to the independent claims, embodiments thereof being defined by the dependent claims.

A first aspect of the invention is a signal filtering device, comprising: an input for receiving at least one pressure signal from a set of pressure sensors in a fluid containing system, the at least one pressure signal comprising first pulses originating from a first pulse generator and second pulses, and a signal processor connected to the input. The signal processor is configured to: populate a plurality of signal vectors of identical length such that each of the signal vectors corresponds to a respective signal segment of signal values in the at least one pressure signal; process the signal vectors by Principal Component Analysis, PCA, so as to compute principal components for the signal vectors; select, among the principal components, either a first subset of principal components that primarily represent the first pulses or a second subset of principal components that are excluded from the first subset; determine a first set of weights for the principal components in the first subset or a second set of weights for the principal components in the second subset; and generate a filtered signal by one of: subtracting, as a function of the first set of weights, the principal components in the first subset from the at least one pressure signal, and combining, as a function of the second set of weights, the principal components in the second subset.

Principal component analysis, PCA, is a statistical procedure that is mostly used as a tool in exploratory data analysis and for making predictive models. Depending on the field of application, PCA is also named the discrete KarhunenLoeve Transform (KLT), the Hotelling Transform, Proper Orthogonal Decomposition (POD), Eckart Young Theorem, Empirical Orthogonal Functions (EOF), and Spectral Decomposition. A common feature of PCA algorithms is that a covariance matrix (or equivalently, a correlation matrix) is estimated for a data set and processed for computation of eigenvectors and eigenvalues, so that principal components and variances can be defined based on the eigenvectors and the eigenvalues, respectively. The principal components are thus uncorrelated and take the form of basis functions for the data set. The variances represent the variability of the data set along the respective principal component.

The first aspect is based on the insight that the principal components obtained by PCA processing may be analyzed for estimating the contribution from the first pulses to the at least one pressure signal, provided that the PCA processing operates on signal vectors that correspond to a respective signal segment in the at least one pressure signal. While the underlying mathematical theory of PCA is complex, there are established and processing-efficient PCA algorithms that may be efficiently implemented in the inventive signal filtering device in either hardware or software, or a combination of thereof, to compute the principal components. The set of weights may be obtained as a by-product of the PCA processing or by simple and standard operations, such as by correlation. The computation of a correlation value may, e.g., be implemented as a simple scalar product (dot product) between two vectors. Likewise, the generation of the filtered signal may be implemented by simple and standard operations. In contrast to prior art approaches using adaptive filters, which are iterative by nature, the first aspect provides a direct approach of determining signal contributions to be subtracted from the pressure signal for the purpose of eliminating or at least significantly suppressing the first pulses. Thus, in contrast to approaches using adaptive filters, the inventive filtering technique has no stability or convergence issues, e.g. after a change in operating frequency for the first pulse generator. Furthermore, the filtering technique of the first aspect may obviate the need to store a library of reference profiles.

The first aspect may be seen to include two mutually exclusive approaches of generating the filtering signal, one approach being based on identifying and subtracting the signal contribution of the first pulses, whereas the other approach is based on directly identifying a signal contribution that excludes the first pulses. The signal filtering device is thus configured to either select the first subset of principal components or the second subset of principal components. If the signal filtering device is configured to select the first subset, the first set of weights is determined and the principal components in the first subset are subtracted from the at least one pressure signal, using the first set of weights. If the signal filtering device is configured to select the second subset, the second set of weights is determined and the principal components in the second subset are combined, using the second set of weights.

If the signal filtering device computes the principal components based on more than one incoming pressure signal, any one of the incoming pressure signals may be filtered by subtracting the principal components in the first subset.

In one embodiment, the signal processor is further configured, when processing the signal vectors by PCA, to: process the signal vectors to compute eigenvectors for an estimated covariance matrix comprising estimated covariance values for the signal vectors, and optionally to compute eigenvalues associated with the eigenvectors, and to obtain the principal components based on the eigenvectors. The estimated covariance matrix may be given by ƒ(X^{T}X), wherein X is a matrix with the signal vectors arranged as rows or columns, X^{T} is a transpose of the matrix X, and ƒ is a linear function. It should be noted that the eigenvectors, and optionally the eigenvalues, may be computed without a need to explicitly populate the estimated covariance matrix. However, the signal processor may be further configured, when processing the signal vectors by PCA, to: compute the estimated covariance values, populate the estimated covariance matrix by the estimated covariance values, and process the estimated covariance matrix for computation of the eigenvectors and, optionally, the eigenvalues. If the signal vector values in the signal vectors are known to have a non-Gaussian distribution, the signal processor may be further configured, when processing the signal vectors by PCA, to: process the eigenvectors to generate a set of mutually independent eigenvectors for the estimated covariance matrix, and obtain the principal components based on the set of mutually independent eigenvectors.

In one embodiment, the first pulse generator is periodic, and the signal processor is configured to populate the signal vectors such that at least a subset of the signal vectors correspond to mutually time-shifted signal segments within a time window in a dedicated pressure signal included among the at least one pressure signal, or within a time window in an intermediate signal generated based on the at least one pressure signal. Thus, in this embodiment, at least a subset of the populated signal vectors correspond to mutually time-shifted signal segments within one and the same signal, which is either a pressure signal or an intermediate signal. If the signal filtering device receives at least two pressure signals, the signal processor may be configured to generate the intermediate signal by linearly combining corresponding pressure values in the at least two pressure signals.

In one embodiment, the signal processor is configured to populate the signal vectors such that said at least a subset of the signal vectors correspond to partly overlapping and mutually time-shifted signal segments in the dedicated pressure signal or the intermediate signal.

In one embodiment, the signal processor may be configured to populate the signal vectors exclusively based on the mutually time-shifted signal segments in the dedicated pressure signal or the intermediate signal.

Alternatively, when the input receives a plurality of pressure signals from a plurality of pressure sensors in the fluid containing system, the signal processor may be configured to populate the signal vectors such that each signal vector corresponds to a respective signal segment in a respective one of the pressure signals.

In one embodiment, the signal processor is configured, when selecting the first or second subset, to: determine a current frequency associated with the first pulses; identify, among the principal components computed by PCA, a group of principal components that have a dominating frequency with a predefined relation to the current frequency; and select the first or second subset based on said group of principal components. The signal processor may be configured to select said group of principal components as the first subset, and/or to the select the second subset among the principal components computed by PCA while excluding said group of principal components.

In one embodiment, the signal processor is configured, when processing the signal vectors by PCA, to: compute variances associated with the principal components, and the signal processor is configured, when determining the current frequency, to: select an initial principal component among the principal components computed by PCA based on the magnitude of the variances associated with the principal components, such that the initial principal component represents the current frequency. In one implementation, in which the first pulses are known to exceed the second pulses in magnitude in the at least one pressure signal and the signal vector values in each of the signal vectors is zero on average, the signal processor is configured to: select, as the initial principal component, the principal component associated with the largest variance among the variances computed by PCA.

In one embodiment, the signal processor is configured, when identifying said group of principal components, to: identify all principal components that have a dominating frequency which is a multiple of the dominating frequency of the initial principal component.

In one embodiment, the first pulse generator comprises a peristaltic pump with a number of rollers, and the signal processor is configured, when identifying said group of principal components, to: identify all principal components that have a dominating frequency which is a multiple of the dominating frequency of the initial principal component divided by the number of rollers.

In one embodiment, the signal processor is configured, when generating the filtered signal, to: subtract, from the at least one pressure signal, a linear combination of the principal components in the first subset, the first set of weights forming coefficients in the linear combination, or linearly combine the principal components in the second subset using the second set of weights as coefficients.

The signal processor may be configured, when processing the signal vectors by PCA, to: compute variances associated with the principal components, and the signal processor may be configured to determine the first or second set of weights as a function of the variances associated with the principal components in the first or second subset, respectively.

Alternatively, the signal processor may be configured, when determining the first or second set of weights, to: compute, for each one of a plurality of signal segments in the at least one pressure signal, a respective set of correlation values estimating correlations between each of the principal components in the first or second subset and the signal vector corresponding to said each one of the plurality signal segments, and determine the first or second set of weights as a function of the respective set of correlation values; and the signal processor may be configured, when generating the filtered signal, to: generate, as a function of the first or second set of weights determined for said each one of the plurality of signal segments, a filtered signal segment, so as to produce a plurality of filtered signal segments for the plurality of signal segments. The signal processor may be configured to generate the filtered signal by concatenating at least parts of the filtered signal segments, and/or by combining overlapping parts of the filtered signal segments.

In one embodiment, the signal processor is further configured, before processing the signal vectors by PCA, to: process the at least one pressure signal and/or the signal vectors to yield an average of zero for the signal vector values in the respective signal vector.

In one embodiment, the signal processor is further configured, before populating the plurality of signal vectors, to filter the at least one pressure signal so as to decrease the first pulses in magnitude in relation to the second pulses.

In one embodiment, the fluid containing system comprises an extracorporeal blood circuit fluidly connected to a vascular system in a human body, and the first pulse generator comprises a pumping device in the extracorporeal blood circuit, and the second pulses originate from a second pulse generator in or attached to the human body.

A second aspect of the invention is a signal filtering device, which comprises: means for receiving at least one pressure signal originating from a set of pressure sensors in a fluid containing system, the at least one pressure signal comprising first pulses originating from a first pulse generator and second pulses; means for populating a plurality of signal vectors of identical length such that each of the signal vectors corresponds to a respective signal segment of signal values in the at least one pressure signal; means for processing the signal vectors by Principal Component Analysis, PCA, so as to compute principal components for the signal vectors; means for selecting, among the principal components, either a first subset of principal components that primarily represent the first pulses or a second subset of principal components that are excluded from the first subset; means for determining a first set of weights for the principal components in the first subset or a second set of weights for the principal components in the second subset; and means for generating a filtered signal by one of: subtracting, as a function of the first set of weights, the principal components in the first subset from the at least one pressure signal, and combining, as a function of the second set of weights, the principal components in the second subset.

A third aspect of the invention is a filtering method performed by a data processor. The method comprises: receiving at least one pressure signal originating from a set of pressure sensors in a fluid containing system, the at least one pressure signal comprising first pulses originating from a first pulse generator and second pulses; populating a plurality of signal vectors of identical length such that each of the signal vectors corresponds to a respective signal segment of signal values in the at least one pressure signal; processing the signal vectors by Principal Component Analysis, PCA, so as to compute principal components for the signal vectors; selecting, among the principal components, either a first subset of principal components that primarily represent the first pulses or a second subset of principal components that are excluded from the first subset; determining a first set of weights for the principal components in the first subset or a second set of weights for the principal components in the second subset; and generating a filtered signal by one of: subtracting, as a function of the first set of weights, the principal components in the first subset from the at least one pressure signal, and combining, as a function of the second set of weights, the principal components in the second subset.

A fourth aspect of the invention is a computer-readable medium comprising processing instructions for causing a data processor to perform the method of the third aspect.

Any one of the above-identified embodiments of the first aspect may be adapted and implemented as an embodiment of the second to fourth aspects.

Still other objectives, features, aspects and advantages of the present invention will appear from the following detailed description, from the attached claims as well as from the drawings.

### BRIEF DESCRIPTION OF DRAWINGS

Embodiments of the invention will now be described in more detail with reference to the accompanying schematic drawings.

FIG. 1 a schematic diagram of a blood path in an extracorporeal blood processing apparatus attached to a human subject.

FIG. 2(a) is a plot in the time domain of a pressure signal containing both pump pulses and heart pulses, and FIG. 2(b) is a plot of the corresponding signal in the frequency domain.

FIG. 3(a) is a side view of a rotor of a peristaltic pump, and FIG. 3(b) is a plot of pressure pulses generated during a full rotation of the rotor in FIG. 3(a), as measured by a pressure sensor in the extracorporeal blood processing apparatus of FIG. 1.

FIGS. 4(a)-(b) are block diagrams of filtering devices that operate on a plurality of pressure signals and a single pressure signal, respectively.

FIG. 5 is a flow chart of an embodiment of a method of generating a filtered signal.

FIG. 6 illustrates how signal vectors are populated by signal segments within a time window in a venous pressure signal, for use in PCA processing.

FIG. 7 illustrates a sequence of principal components ordered by decreasing magnitude of the associated variance and obtained by PCA processing of the signal vectors in FIG. 6.

FIGS. 8(a)-(b) illustrate different techniques of subtracting a pump profile from the pressure signal in FIG. 6.

FIGS. 9(a)-9(b) are block diagrams of filtering devices that generate a filtered signal by subtraction of principal components, and FIGS. 9(c)-9(d) are block diagrams of a filtering devices that generate a filtered signal by combination of principal components.

FIGS. 10(a)-10(b) are plots of a venous pressure signal, a pump profile signal and a filtered signal, with and without pulsations originating from the subject in FIG. 1.

### DETAILED DESCRIPTION OF EXAMPLE EMBODIMENTS

Throughout the description, the same reference numerals are used to identify corresponding elements.

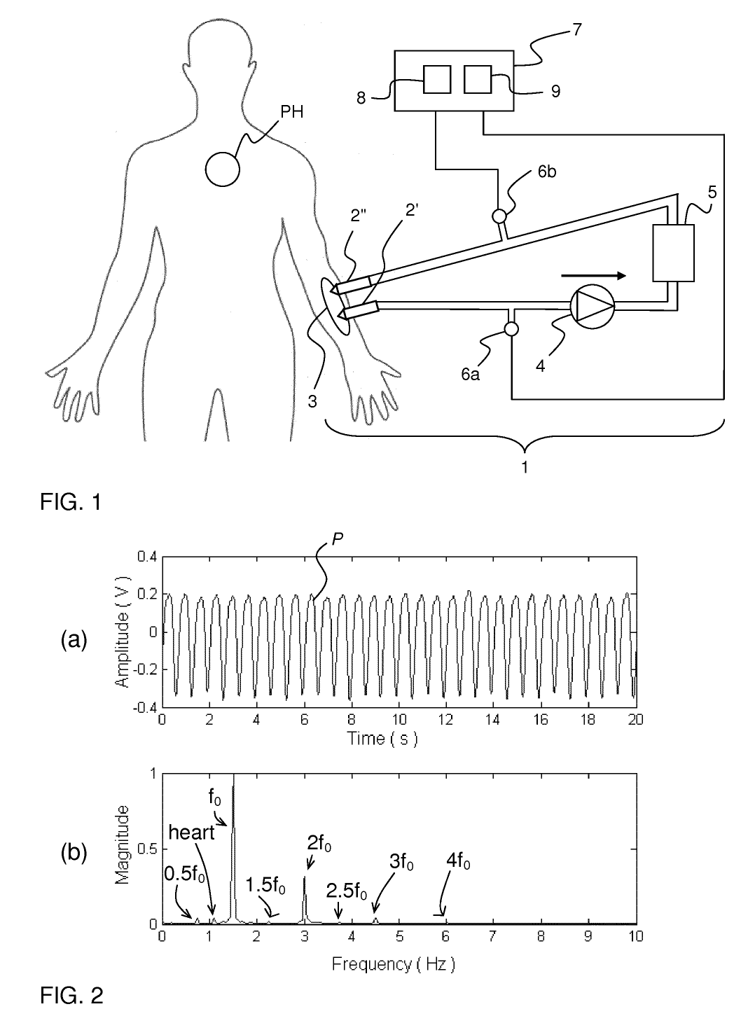

FIG. 1 illustrates a human subject which is connected to an extracorporeal fluid circuit 1 by way of access devices 2′, 2″ inserted into a dedicated vascular access 3 (also known as “blood vessel access”) on the subject. The extracorporeal fluid circuit 1 (denoted “EC circuit” in the following) is configured to communicate blood to and from the cardiovascular system of the subject. In one example, the EC circuit 1 is part of an apparatus for blood processing, such as a dialysis machine. In the illustrated example, a blood pump 4 draws blood from the vascular access 3 via access device 2′ and pumps the blood via blood lines (tubes) through a blood processing unit 5, e.g. a dialyzer, and back to the vascular access 3 via access device 2″. Thus, when both access devices 2′, 2″ are connected to the vascular access 3, the EC circuit 1 defines a blood path that starts and ends at the vascular access 3. The EC circuit 1 may be seen to comprise a “venous side” which is the part of the blood path located downstream of the blood pump 4, and an “arterial side” which is the part of the blood path located upstream of the pump 4. Pressure sensors 6a and 6b are arranged to detect pressure waves in the EC circuit 1. As used herein, a “pressure wave” is a mechanical wave in the form of a disturbance that travels or propagates through a material or substance. In the context of the following examples, the pressure waves propagate in the blood in the cardiovascular system of the subject and in the blood path of the EC circuit 1 at a velocity that typically lies in the range of about 3-20 m/s. The pressure sensors 6a, 6b, which are in direct or indirect hydraulic contact with the blood, generate pressure data that forms a pressure pulse for each pressure wave. A “pressure pulse” is thus a set of data samples that define a local increase or decrease (depending on implementation) in signal magnitude within a time-dependent measurement signal (“pressure signal”).

The pressure sensors 6a, 6b may be of any type, e.g. operating by resistive, capacitive, inductive, magnetic, acoustic or optical sensing, and using one or more diaphragms, bellows, Bourdon tubes, piezo-electrical components, semiconductor components, strain gauges, resonant wires, accelerometers, etc. For example, the pressure sensors 6a, 6b may be implemented as a conventional pressure sensor, a bioimpedance sensor, or a photoplethysmography (PPG) sensor.

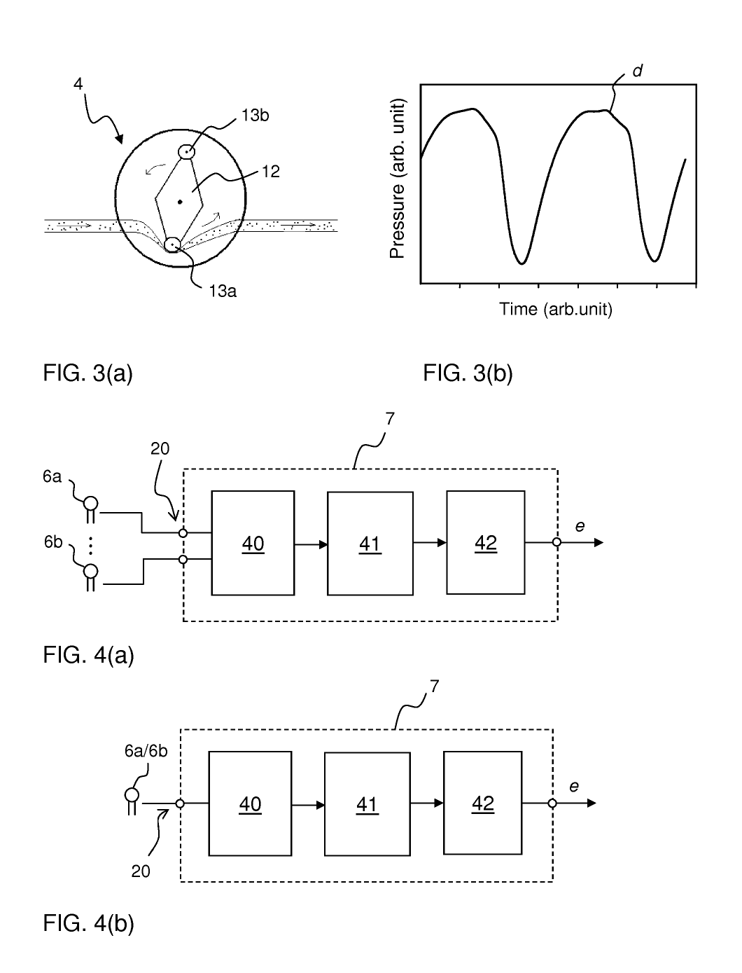

FIG. 2(a) shows an example of a time-resolved pressure signal P acquired from sensor 6b in FIG. 1, and FIG. 2(b) shows the corresponding spectral density, i.e. signal energy as a function of frequency. The spectral density reveals that the pressure signal P contains frequency components that emanate from and are given by the design of the blood pump 4. As seen, the frequency components are a set of harmonic frequencies 0.5f₀, f₀, 1.5f₀, 2f₀, etc. In the illustrated example, the blood pump 4 is a rotary peristaltic pump of the type indicated in FIG. 3(a), and the frequency components are governed by the revolution of the rotor 12 and the engagement of the rollers 13a, 13b with the tube segment. The dominating frequency f₀ is the pumping frequency, i.e. the frequency of pump strokes, with each pump stroke being generated by the engagement of one of the rollers 13a, 13b with the tube segment. FIG. 3(b) illustrates the pressure pulsations (“pump pulses”, d) in the pressure signal that originate exclusively from the pump 4 during one revolution of the rotor 12. Thus, the pump pulses din FIG. 3(b) represent the pressure waves that are generated by the rollers 13a, 13b engaging the tube segment during a full rotor revolution. Returning to FIGS. 2(a)-2(b), the pressure signal P also includes pressure pulsations (“heart pulses”) that originate from the beating of the heart in the patient. In this example, the heart pulses are much weaker than the pump pulses and are difficult to detect in the pressure signal P, which is dominated by the pump pulses. Generally, the pressure signal P may contain pressure pulses from any physiological pulse generator PH (FIG. 1), periodic or non-periodic, in the patient, including reflexes, voluntary muscle contractions, non-voluntary muscle contractions, the heart, the breathing system, the autonomous system for blood pressure regulation and the autonomous system for body temperature regulation. In a further alternative, the pressure signal P may contain pressure pulses from a dedicated pulse generator attached to the patient. In the following, pressure pulses originating from the patient are collectively denoted “patient pulses”.

Returning to the specific example in FIG. 1, a filtering device 7 is connected to the pressure sensors 6a, 6b by a respective transmission line. In the following, the pressure sensors 6a, 6b are denoted “arterial pressure sensor” and “venous pressure sensor”, respectively, and generate an arterial pressure signal and a venous pressure signal. The device 7 is operable to acquire and process at least one of the pressure signals from the arterial and venous pressure sensors 6a, 6b for the purpose of generating a filtered signal which contains patient pulses from at least one pulse generator in or attached to the patient while the pump pulses are eliminated or at least significantly suppressed in relation to the patient pulses. Embodiments of the invention may e.g. be implemented by software instructions that are supplied on a computer-readable medium for execution by a processor 8 in conjunction with an electronic memory 9 in the device 7.

It is realized that the filtered signal may be further processed, by the device 7 or a separate device, for any type of monitoring purpose, e.g. as described in the Background section. Such monitoring purposes include monitoring the integrity of the connection between the EC circuit 1 and the patient, e.g. with respect to VND or proper placement of the access devices 2′, 2″, and monitoring/analyzing the behavior of physiological pulse generators PH in the patient, such as the heart or the respiratory system.

Depending on monitoring purpose, the filtered signal may be generated either to represent the patient pulses in a specific pressure signal, or to represent the general appearance of the patient pulses in all of the pressure signals that are processed for generating the filtered signal. For VND monitoring, the filtered signal may be generated to represent the patient pulses in the venous pressure signal. For monitoring proper placement of the access devices 2′, 2″, two filtered signals may be generated; one representing the patient pulses in the venous pressure signal and the other representing the patient pulses in the arterial pressure signal. For monitoring/analyzing the behavior of physiological generators in the patient, the filtered signal may be generated to either represent the patient pulses in a specific pressure signal, or represent the general appearance of the patient pulses in all of the pressure signals being processed.

The signal processing in the device 7 is based on the insight that Principal Component Analysis (PCA) may be utilized for separating and removing the pump pulses from the pressure signals. It should be understood that each of the venous and arterial pressure signals includes a mixture of pressure pulses originating from a number of different pulse generators. In practice, the same pressure pulses show up in both of the venous and arterial pressure signals, approximately at the same time, although the shape and magnitude of the pressure pulses may differ between the signals. Thus, the pressure signals are correlated.

PCA is a statistical procedure that uses an orthogonal linear transformation to convert a set of observations of correlated variables into a set of values of linearly uncorrelated variables called “principal components”. The number of principal components is less than or equal to the number of original variables. This transformation is defined in such a way that the first principal component has the largest possible variance, i.e. accounts for as much of the variability in the data as possible, and each succeeding component in turn has the highest variance possible under the constraint that it is orthogonal to (i.e., uncorrelated with) the preceding components. Thus, PCA processing results in a number of principal components for the set of observations, and may also result in a variance for each principal component.

The device 7 is configured to select a set of “observations” among the pressure values in one or more pressure signals, process the set of observations by PCA, and generate the filtered signal by extracting and processing a carefully selected subset of the principal components resulting from the PCA processing.

FIG. 4(a) illustrates an embodiment of the device 7 configured to process a plurality of pressure signals, including or consisting of the venous and arterial pressure signals from the sensors 6a, 6b, for the purpose of generating a filtered signal e that represents the appearance of the patient pulses in one of these pressure signals. The device 7 contains an input interface 20 for the pressure signals and an extraction module 40 connected to the input interface 20. The extraction module 40 is configured to define the above-mentioned set of observations by extracting pressure values from the pressure signals and populating a set of signal vectors based on the extracted pressure values. Specifically, the module 40 populates one signal vector for each pressure signal. A PCA module 41 is configured to process the signal vectors by PCA to generate a number of principal components, and optionally a corresponding number of variances. A filtering module 42 selects a subset of the principal components and generates the filtered signal e based on the subset. In principle, two pressure signals are needed to separate pressure pulses from two different pulse generators. The quality of the filtered signal e generally improves with the number of pressure signals that are included in the set of signal vectors. The embodiment in FIG. 4(a) has the ability to remove or suppress pulses that occur either periodically (regularly) or non-periodically (irregularly) in the pressure signal. Thus, this embodiment may not only be used for removing pump pulses, which typically are periodic, but also other interference pulses that are less regular, such as pulses caused by switching of valves in the EC circuit 1.

FIG. 4(b) illustrates an embodiment of the device 7 configured to generate a filtered signal e that represents the patient pulses in either the venous pressure signal or the arterial pressure signal. The device in FIG. 4(b) includes the same modules 40-42 as the device in FIG. 4(a), but module 40 is configured to populate the set of signal vectors by extracted pressure values from a single pressure signal. Specifically, the signal vectors are populated to correspond to a respective segment of consecutive pressure values in the pressure signal. Thus, the set of signal vectors to be processed by PCA corresponds to time-shifted segments in a single pressure signal. These signal vectors are correlated, since the same pressure pulses show up in all or at least part of the segments, depending on the length of the segments. Preferably, each segment has a sufficient length to contain a plurality of pump pulses, when present in the pressure signal. The PCA module 41 and the filtering module 42 may be identical to the modules 41, 42 in FIG. 4(a). The embodiment in FIG. 4(b) is operable to remove or suppress pulses that occur periodically in the pressure signal.

In a variant, which is a combination of the foregoing embodiments, the module 40 in FIG. 4(a) is configured to populate more than one signal vector based on a respective one of the pressure signals, by applying the technique used by the module 40 in FIG. 4(b). Thus, in this variant, a plurality of signal vectors are populated to correspond to time-shifted segments in one and the same pressure signal. For example, the module 40 may populate the signal vectors to correspond to more than one signal segment in each of the pressure signals. Generally, this variant may improve the quality of the filtered signal e, compared to the embodiment in FIG. 4(a), for a given number of pressure signals. However, the computational load and memory requirement may increase with the number of signal vectors.

In yet another variant, the module 40 in FIG. 4(a) is configured to generate an intermediate signal by linearly combining two or more pressure signals, and to populate the signal vectors to correspond to a plurality of time-shifted signal segments in the intermediate signal. The intermediate signal may be generated as a linear combination of pressure values in overlapping (time-synchronized) signal segments in the two or more pressure signals. The intermediate signal is thus an artificial pressure signal that comprises a time-sequence of pressure values and is generated for the purpose of populating the signal vectors.

In the embodiments of FIGS. 4(a)-4(b), the filtered signal e is output from the device 7 via an output interface (not shown). Alternatively or additionally, device 7 may include a monitoring module (not shown) which is configured to process the filtered signal e for a dedicated monitoring purpose.

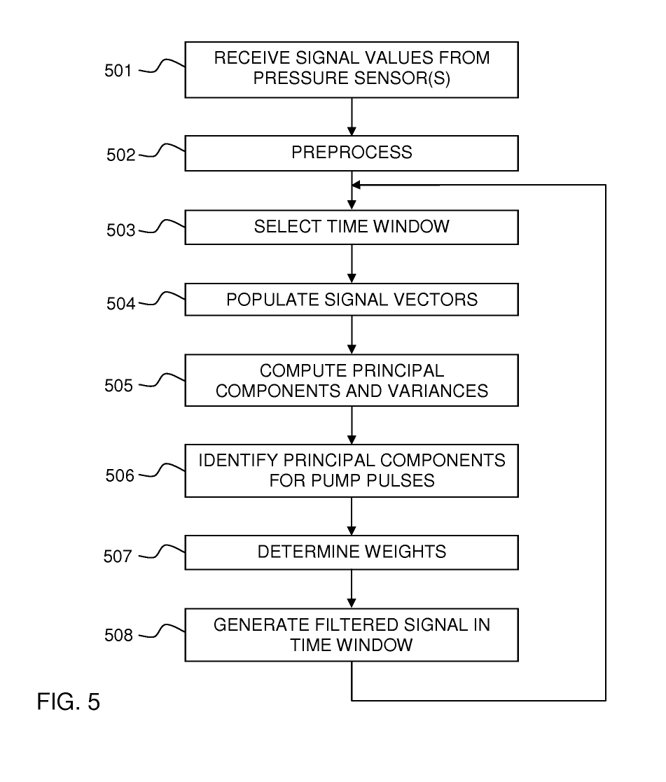

More detailed examples of the inventive filtering technique will now be described with reference to the flow chart in FIG. 5, which illustrates a filtering method involving a sequence of steps that may be implemented by the device 7 shown in FIGS. 4(a)-4(b). For clarity of presentation, the flow chart will first be presented when operating on a single pressure signal, e.g. for the process in the device 7 of FIG. 4(b).

The filtering method involves a step 501 that receives a pressure signal from a pressure sensor, and a step 502 that pre-processes the pressure signal, e.g. for AD conversion, signal amplification, removal of offset, high frequency noise and supply voltage disturbances, etc. The removal of offset may be implemented to ensure that the pre-processed pressure signal has a zero mean, which may be advantageous for the subsequent PCA processing. Step 502 may also include a pre-filtering of the pressure signal, e.g. to suppress the pump pulses in relation to the patient pulses. Any known filtering technique may be used, e.g. any one of the techniques disclosed in WO97/10013, US2005/0010118, WO2009/156175, WO2010/149726, WO2010/149726, WO2013/000777, WO2014/009111, and WO2015/032948. Steps 501 and 502 may operate to continuously supply pre-processed pressure values, independently of the other steps of the method. In FIGS. 4(a)-4(b), steps 501 and 502 may be implemented by a combination of the interface 20 and the module 40. In certain embodiments, step 502 may be omitted.

The method further includes a repeating sequence of steps 503-508 that operate on a respective time window in the pressure signal that is supplied by steps 501-502, to generate a filtered signal that corresponds to the pressure values in the time window. The consecutive time windows processed by steps 503-508 may be overlapping or non-overlapping.

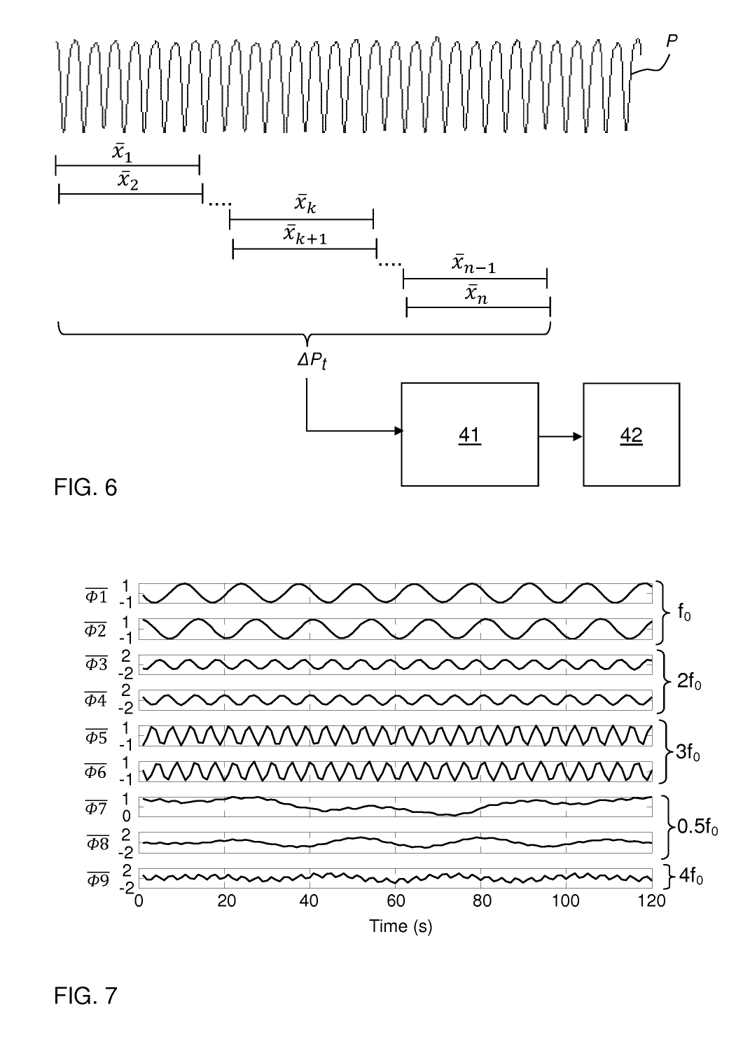

In one repetition of steps 503-508, step 503 first selects a time window in the pressure signal. Such a time window ΔPₜ is shown in FIG. 6 in relation to the pressure signal P in FIG. 2(a). The length of the time window ΔPₜ is predefined and set with respect to the number, length and time-shift of the signal vectors obtained in step 504. The following examples utilize a time window of 20 seconds, corresponding to 200 pressure values (data samples), i.e. a sampling rate of 10 Hz. Step 504 populates a set of signal vectors such that each signal vector corresponds to a respective signal segment within the time window ΔPₜ. In FIG. 6, there are n signal vectors, each designated by x̅ₖ, k=1, . . . , n, which correspond to a predefined set of n time-shifted and mutually overlapping signal segments, indicated by horizontal bars in FIG. 6. Each signal vector contains m signal values:

$${\overline{x}}_{k} = [ \begin{matrix}{x}_{k} ( 1 ) \\ {x}_{k} ( 2 ) \\ ⋮ \\ {x}_{k} ( m )\end{matrix} ] .$$

In all examples given herein, it is assumed that the signal vectors are populated by a respective signal segment, i.e. the signal values xₖ(i) of the respective signal vector x̅ₖ are directly given by the pressure values in the signal segment. However, other alternatives are conceivable. For example, each signal value xₖ(i) may be given by grouping and combining pressure values in the signal segment, e.g. a sum, a product or an average of a number of consecutive pressure values. In the following examples, each signal vector corresponds to a signal segment of 12 seconds in the pressure signal, and thus m=120 (given the sampling rate of 10 Hz), and consecutive signal segments are displaced by one data sample within the time window ΔPₜ. Thus, the number of signal vectors x̅ₖ is 81 (=n), and each signal vector x̅ₖ is given by:

$${\overline{x}}_{k} = [ \begin{matrix}P ( k ) \\ P ( k + 1 ) \\ ⋮ \\ P ( k + m - 1 )\end{matrix} ] k = 1 , 2 , … , n$$

where P(k) denotes the pressure value at position k within the time window.

Step 505 processes the signal vectors x̅ₖ by PCA to compute a set of principal components, each denoted by Ψ̅ᵢ, and a variance σᵢ² associated with each principal component. Theoretically, PCA involves applying an orthogonal linear transformation to a data set, i.e. the set of signal vectors {x̅ₖ} which are at least partially correlated, to generate a set of transformed signal vectors that are uncorrelated. The thus-transformed signal vectors are the principal components of the set of signal vectors {x̅ₖ}. As used herein, a variable enclosed in { } indicates a set of values for the variable.

It can be shown that finding the transformed signal vectors is equivalent to finding the eigenvectors of the covariance matrix for the set of signal vectors. The covariance matrix may be estimated for the set of signal vectors by forming a data matrix X in which the signal vectors x̅ₖ are arranged as rows:

$$X = [ \begin{matrix}{\overline{x}}_{1}^{T} \\ {\overline{x}}_{2}^{T} \\ ⋮ \\ ⋮ \\ {\overline{x}}_{n}^{T}\end{matrix} ]$$

and by evaluating the matrix operation X^{T}·X, where superscript T indicates a transpose. The estimated covariance matrix Ĉ is given by ƒ(X^{T}·X), where ƒ is any suitable linear function. For example, the function ƒ may be designed to normalize X^{T}·X in proportion by the number of signal vectors, e.g. through a division by n or n−1. The data matrix X has size n x m (number of rows times number of columns), and the estimated covariance matrix Ĉ contains m x m estimated covariance values. It can be noted that X^{T}·X corresponds to the element-wise sum of the auto-correlations for the respective signal vector x̅ₖ, given by

$${\sum }_{k = 1}^{n} {\overline{x}}_{k} \cdot {\overline{x}}_{k}^{T} .$$

This type of estimated covariance matrix Ĉ enables computation of a maximum of n (if n<m) or m (if m<n) eigenvectors having a respective length of m signal values. In an alternative implementation, the data matrix X is formed by arranging the signal vectors x̅ₖ as columns: X=[x̅₁ x̅₂ . . . x̅ₙ], where X has size m x n. The estimated covariance matrix Ĉ may still be given by ƒ(X^{T}·X) and has size n x n. This type of estimated covariance matrix Ĉ enables computation of a maximum of n (if n<m) or m (if m<n) eigenvectors having a respective length of n signal values. It should be noted that while the foregoing techniques of defining the estimated covariance matrix Ĉ are believed to be straightforward and sufficiently accurate, there are more advanced techniques that may be employed, including regularized or shrinkage estimators.

Step 505 may thus involve computing a set of eigenvectors and eigenvalues for the estimated covariance matrix Ĉ, which is formed based on the signal vectors x̅ₖ that were populated by step 504. The eigenvectors and eigenvalues may be computed using any known technique. In one example, step 505 computes the matrix V of eigenvectors which diagonalizes the estimated covariance matrix Ĉ:

V⁻¹·C·V=D,

where D is the diagonal matrix of eigenvalues of Ĉ. The column vectors of the matrix V represent the eigenvectors of the estimated covariance matrix Ĉ. The eigenvalues and eigenvectors are ordered and paired, i.e. the jth eigenvalue in D corresponds to the jth eigenvector in V. The computation of V and D typically involves the use of a dedicated computer-based algorithm for computing or numerically estimating eigenvectors and eigenvalues. In another example, which obviates the need to explicitly calculate the estimated covariance matrix Ĉ, the eigenvectors and eigenvalues are computed by the singular value decomposition (SVD) of X, as is well-known in the art.

The principal components and variances are directly given by the eigenvectors and eigenvalues. However, it is conceivable that the eigenvectors and eigenvalues are further processed by step 505 to produce the set of principal components {Ψ̅ᵢ} and a set of associated variances {σᵢ²}, e.g. by scaling the eigenvectors and/or the eigenvalues. In one further example, the set of eigenvectors are subjected to a transformation operation that makes the eigenvectors independent of each other, and the principal components are given by the resulting independent (and uncorrelated) eigenvectors. Such a transformation operation is well-known to the skilled person and makes sense to implement when the signal vectors x̅ₖ have a non-Gaussian distribution. The combination of PCA processing and the transformation operation is also known as Independent Component Analysis, ICA.

It should be noted that step 505 need not compute all possible eigenvectors for the estimated covariance matrix Ĉ. The number of eigenvectors to be computed may be predefined and set to give an adequate quality of the filtered signal e, given by previous testing and/or simulation.

As an alternative to processing the pressure signal in step 502 so that it has a zero mean, step 505 may include an initial sub-step that adjusts the signal values of the signal vectors x̅ₖ to a zero mean before they are subjected to PCA processing. This sub-step may involve computing a mean value for the respective signal vector x̅ₖ and subtracting this mean value from the signal values in the signal vector x̅ₖ. Alternatively, a single mean value may be computed for all signal vectors x̅ₖ or for the pressure values within the time window ΔPₜ, and subtracted.

Step 506 operates on the principal components {Ψ̅ᵢ} and the associated variances {σᵢ²} produced by step 505, for the purpose of identifying and selecting a subset of principal components {Ψ̅ᵢ}ₚ that primarily represents the pump pulses. To facilitate the following discussion, FIG. 7 illustrates a set of 9 principal components Ψ̅₁, . . . , Ψ̅₉ computed for the segments in FIG. 6. The principal components Ψ̅₁, . . . , Ψ̅₉ are ordered by magnitude of the associated variance, from top to bottom in FIG. 7. The variance indicates the energy content in the respective principal component, and the ordering of the principal components by variance therefore represents their relative importance for the appearance of the pressure signal within the time window. The pressure sensors 6a, 6b are located in the EC circuit 1, in relative close vicinity to the pump 4, which in itself produces strong pressure waves. Therefore, the pump pulses will generally dominate the pressure signal and be much larger in magnitude than the heart pulses. This means that the principal components with the largest variances are most likely to mainly represent the pump pulses, provided that the pressure signal has a zero mean. If PCA processing is performed on an estimated correlation matrix Ĉ obtained from signal vectors x̅ₖ with non-zero mean, the mean is likely to be reflected in the most significant principal component(s).

Step 506 is designed to identify the most significant principal component for the pump pulses among the principal components, based on the variances associated with the principal components. If the signal vectors x̅ₖ have zero mean, the most significant principal component for the pump pulses has the largest variance. Thus, step 506 selects one of the principal components based on variance. In the following, this principal component is denoted “initial principal component” and represents a current frequency associated with the pump pulses. In FIG. 7, the initial principal component is Ψ̅₁, which represents the current frequency f₀. Based on Ψ̅₁, step 506 then identifies all principal components that primarily represent the pump pulses. Reverting to FIG. 2(b), it should be recalled that while the pump pulses are dominated by the pumping frequency f₀, they also contain harmonics of this frequency at 2f₀, 3f₀, etc. Depending on the design of the pump 4, the pump pulses may also contain at least one smaller frequency given by f₀ divided by the number of rollers 13a, 13b, and harmonics thereof. In FIG. 2(b), the smaller frequency is 0.5f₀, and its harmonics are f₀, 1.5f₀, 2f₀, etc. Thus, all principal components that primarily represent the pump pulses will have a dominating frequency equal to a multiple of 0.5f₀ (when the pump has two rollers). As seen in FIG. 7, all of the illustrated principal components Ψ̅₁-Ψ̅₉ have such a dominating frequency. Although not shown in FIG. 7, it should be understood that step 505 may also compute principal components that represent patient pulses. In the illustrated example, the patient pulses are much weaker than the pump pulses, and the principal components for the patient pulses do not show up among the strongest principal components Ψ̅₁-Ψ̅₉.

Generally, step 506 may select the subset {Ψ̅ᵢ}ₚ to consist of all principal components that have a dominating frequency equal to a multiple of 0.5f₀. As used herein, a “multiple” is any positive integer number. In an alternative, to reduce computational load, step 506 may include in the subset {Ψ̅ᵢ}ₚ only the principal components that have a dominating frequency equal to a multiple of f₀. It is also conceivable, to reduce computation load, that step 506 includes an initial sub-step that screens the principal components based on variance, and eliminates from further processing the principal components that are associated with a variance which falls below a predefined threshold.

There are many ways for step 506 to identify the principal components to be included in the subset {Ψ̅ᵢ}ₚ.

In one implementation, step 506 determines the dominating frequency for each principal component, and identifies all principal components that have a proper dominating frequency (given by its relation to the dominating frequency of the initial principal component). The dominating frequency may, e.g., be determined or estimated by one of: 1) locating the zero crossings in the respective principal component and deriving the mean value of the inverse of the distance between the zero-crossings, 2) locating maximum extreme points (peaks) in the respective principal component and derive the mean value of the inverse of the distance between the maximum extreme points, and 2) locating minimum extreme points in the respective principal component and derive the mean value of the inverse of the distance between the minimum extreme points. In a variant, the dominating frequency is determined as an average of the mean values computed by any combination of the approaches 1)-3).

In another implementation, step 506 identifies the principal components to be included in the subset {Ψ̅ᵢ}ₚ by cross-correlating different time-scaled versions of the initial principal component with each of the other principal components, and selects the principal component having the largest cross-correlation for each time-scaled version. Specifically, to find a certain multiple of the initial principal component among the principal components, the initial principal component is scaled in time in proportion to the multiple. For example, if the principal components related to 2f₀ should be identified, the initial principal component is scaled down in time (resampled) by a factor of two (i.e. its frequency is doubled). Then, a cross-correlation is calculated between this time-scaled version and the other principal components. The maximum correlation values from each cross-correlation are compared to each other, and the principal components corresponding to the two largest correlation values are selected to be included in the subset {Ψ̅ᵢ}ₚ. It is realized that this implementation will identify the principal components that represent the pump pulses without the need to explicitly estimate any dominating frequencies in the principal components. Nevertheless, step 506 indirectly determines a current frequency associated with the pump pulses, by identifying the initial principal component, and selects the subset {Ψ̅ᵢ}ₚ based thereon. It should also be noted that the cross-correlation may be implemented by evaluating scalar products between vectors, which is a processing-efficient operation.

In addition to the subset {Ψ̅ᵢ}ₚ, step 506 also identifies the variances that are associated with the principal components in the subset {Ψ̅ᵢ}ₚ and forms a corresponding subset of variances {σᵢ²}ₚ.

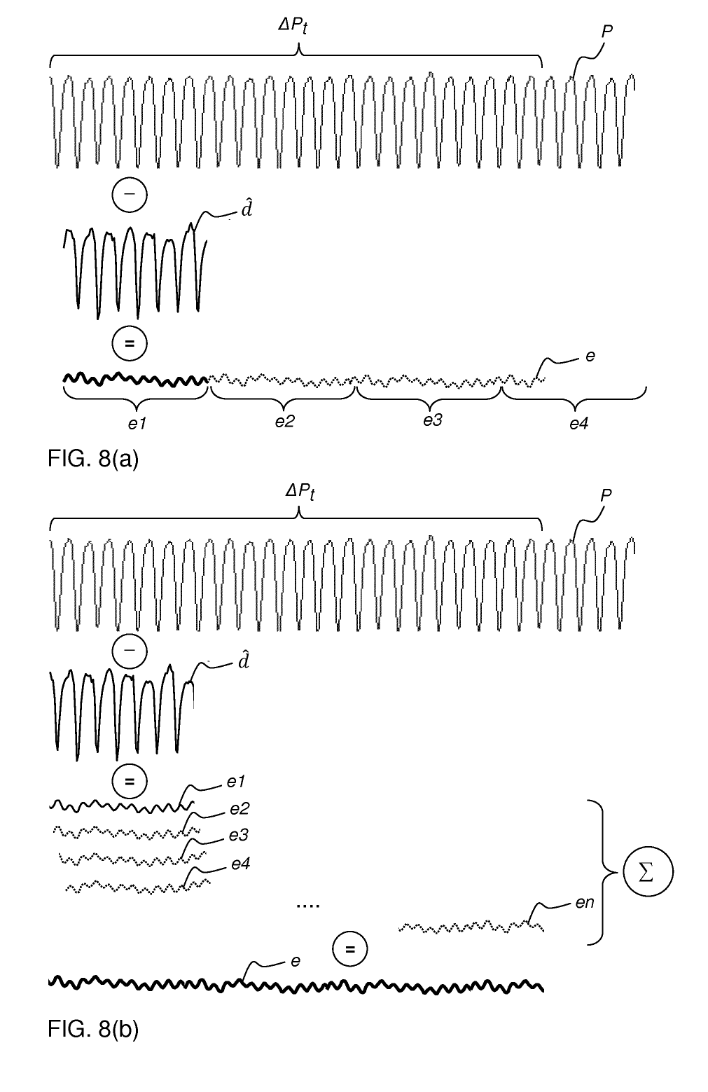

Step 507 determines a set of weights {wᵢ}ₚ to be used when combining the principal components in the subset {Ψ̅ᵢ}ₚ in step 508. Step 507 thereby generates at least one weight wᵢ for each principal component Ψ̅ᵢ in the subset {Ψ̅ᵢ}ₚ. In a first implementation, the weights {wᵢ}ₚ are given by the variances in the subset {σᵢ²}ₚ, either directly or after a predefined modification of the respective variance, e.g. a re-scaling. In a second implementation, which does not require step 507 to select and provide the subset {σᵢ²}ₚ, each weight wᵢ is generated as a correlation value resulting from a correlation of each principal component in the subset {Ψ̅ᵢ}ₚ with one or more of the signal segments in the time window (cf. FIG. 6). The second implementation is described in more detail further below with reference to FIG. 8(b).

Step 508 generates the filtered signal e by combining the principal components in the subset {Ψ̅ᵢ}ₚ into an “estimated pump profile” \hat{d}, i.e. a signal that represents the pump pulses within the time window ΔPₜ (FIG. 6), and then subtracting the estimated pump profile \hat{d} from the pressure signal P within the time window ΔPₜ. The estimated pump profile \hat{d} is formed as a linear combination of the principal components, which are scaled by its associated weight, i.e. the weights are used as coefficients in the linear combination:

$$\hat{d} = {\sum }_{i = 1}^{s} {w}_{i} \cdot {\overline{Φ}}_{i}$$

where Ψ̅ᵢ is a principal component in the subset {Ψ̅ᵢ}ₚ and s is the number of principal components in the subset {Ψ̅ᵢ}ₚ.

There are two implementations of step 508, depending on the weights that are generated by step 507.

A first implementation of step 508 is illustrated in FIG. 8(a) and is denoted “averaged implementation” in the following. Here, the weights correspond to the variances for the principal components in the subset {Ψ̅ᵢ}ₚ, and therefore the estimated pump profile \hat{d} is an averaged pump profile which is applicable to all segments within the time window ΔPₜ. Such a pump profile \hat{d} is not fitted in phase with the pressure signal P. The correct phase may be obtained by cross-correlating the pump profile \hat{d} with a first portion of the pressure signal P, where the phase shift that corresponds to the maximum correlation value gives the correct phase. Instead of using cross-correlation, a “best fit” may be obtained by finding the smallest error (e.g. mean square error) between the pump profile \hat{d} and the first portion of the pressure signal P. The correct phase is obtained from the phase shift that produces the smallest error. The amplitude/magnitude of the pump profile \hat{d} may also be adjusted/scaled, e.g. by multiplying the pump profile \hat{d} with a correction value. The correction value may be determined in the same way as the correct phase. Then, the pump profile \hat{d} is adjusted in phase, and optionally also in amplitude, and subtracted from the pressure signal in order to remove/eliminate the pressure variations from the blood pump. This results in a first filtered segment e1, as shown in FIG. 8(a). The procedure of finding a proper phase, and possibly amplitude, is then repeated for subsequent portions of the pressure signal P, within the time window ΔPₜ, and a properly adjusted pump profile \hat{d} is subtracted from each such portion. The result is an additional number of filtered signal segments, indicated by e2, e3 and e4 in FIG. 8(a). The filtered signal segments e1-e4 are then concatenated to form the filtered signal e.

A second implementation of step 508 is shown in FIG. 8(b) and is denoted “fitted implementation” in the following. Here, the weights are given by the correlation values that were computed in step 507, according to the second implementation thereof. Assuming that step 507 computes a group of weights for each signal segment by correlating each principal component in the subset {Ψ̅ᵢ}ₚ with the signal segment, an estimated pump profile \hat{d} may be calculated for each signal segment and will automatically be tailored to this signal segment with respect to both phase and amplitude. The estimated pump profile is thus a “fitted pump profile”. The fitted pump profile \hat{d} may be created with a maximum length of m (assuming that X has size n x m) and minimum length corresponding to the longest period time (smallest frequency) of the principal components in the subset {Ψ̅ᵢ}ₚ. The fitted pump profile \hat{d} is subtracted from the signal segment to yield a filtered signal segment e1. The procedure of creating the fitted pump profile \hat{d} may be repeated for each signal segment within the time window ΔPₜ, using the weights determined in step 507 for the respective signal segment, and the fitted pump profile \hat{d} is subtracted from the respective signal segment to yield further filtered signal segments, indicated by e2, e3, e4, . . . , en in FIG. 8(b).

The filtered signal segments e1-en may then be combined to form the filtered signal e, e.g. by calculating a (weighted) sum or average of the data samples in the filtered signal segments e1-en for each time step within the time window ΔPₜ. This calculation is indicated by the encircled Σ in FIG. 8(b). To reduce the computation load, a sparser set of filtered signal segments may be generated, e.g. by creating and subtracting the fitted pump profile \hat{d} for only a subset of the signal segments, e.g. every jth signal segment. The filtered signal e may be computed by concatenating non-overlapping portions and/or combining overlapping portions of the filtered signal segments. In one extreme, the fitted pump profile \hat{d} is created for and subtracted from a selected set of non-overlapping signal segments that are located adjacent to one another along the time window ΔPₜ, similar to the set of filtered signal segments e1-e4 shown at the bottom of FIG. 8(a). It is realized that step 507 only needs to generate and supply a respective set of weights for the signal segments that are processed by subtraction in step 508.

As an alternative to generating the filtered signal e by subtraction of the subset {Ψ̅ᵢ}ₚ, consisting of the principal components that are found to primarily represent the pump pulses, the filtered signal e may instead be generated by combining the remaining principal components, i.e. by identifying a residual subset {Ψ̅ᵢ}ᵣ of principal components, {Ψ̅ᵢ}ᵣ={Ψ̅ᵢ}−{Ψ̅ᵢ}ₚ. Alternatively, only some of the remaining principal components are included in the residual subset {Ψ̅ᵢ}ᵣ. The principal components in the residual subset {Ψ̅ᵢ}ᵣ represents the heart pulses, and all other pulsations in the pressure signal, periodic or not, but excludes most of the pump pulses (which are represented by the subset {Ψ̅ᵢ}ₚ). Such an alternative embodiment may be implemented without changing steps 501-505 in FIG. 5. Step 506 is modified to include a final sub-step of selecting the residual subset {Ψ̅ᵢ}ᵣ to consist of the principal components that are not included in the subset {Ψ̅ᵢ}ₚ. Step 506 also identifies the variances that are associated with the principal components in the subset {Ψ̅ᵢ}ᵣ and forms a corresponding subset of variances {σᵢ²}ᵣ. Step 507 is carried out as described above, according to the first (“averaged”) or second (“fitted”) implementation, with the difference that step 507 operates on the subsets {Ψ̅ᵢ}ᵣ and {σᵢ²}ᵣ to generate a set of weights {wᵢ}ᵣ. Step 508 generates the filtered signal e by combining the principal components in the subset {Ψ̅ᵢ}ᵣ. The filtered signal e is formed as a linear combination of the principal components, which are scaled by weight, i.e. the weights are used as coefficients in the linear combination:

$$e = {\sum }_{i = 1}^{s} {w}_{i} \cdot {\overline{Φ}}_{i}$$

where Ψ̅ᵢ is a principal component in the subset {Ψ̅ᵢ}ᵣ and s is the number of principal components in the subset {Ψ̅ᵢ}ᵣ. In analogy with the averaged and fitted implementations described above in relation to step 508, the weights may either correspond to the variances for the principal components in the subset {Ψ̅ᵢ}ᵣ or be given by correlation values computed in step 507. In the averaged implementation of step 508, with weights corresponding to variances, the principal components in the subset {Ψ̅ᵢ}ᵣ are linearly combined using the weights to produce a filtered signal segment which is an average representation for the time window ΔPₜ. It is to be understood that the filtered signal segment e is smaller than the time window ΔPₜ and is not fitted in phase with the pressure signal P. A “down-sampled” version of the true filtered signal e may be formed by concatenating the filtered signal segment that is computed by step 508 for each time window ΔPₜ in the pressure signal P. In the fitted implementation of step 508, with weights given by correlation values, the principal components in the subset {Ψ̅ᵢ}ᵣ are linearly combined using the weights for each (or a selected subset) of the signal segments within the time window (cf. FIG. 8(b)) to produce a set of partially overlapping or non-overlapping filtered signal segments, similarly to the filtered signal segments shown in FIG. 8(b). The filtered signal segments e1-en may then be combined and/or concatenated to form the filtered signal e, as described in the foregoing.

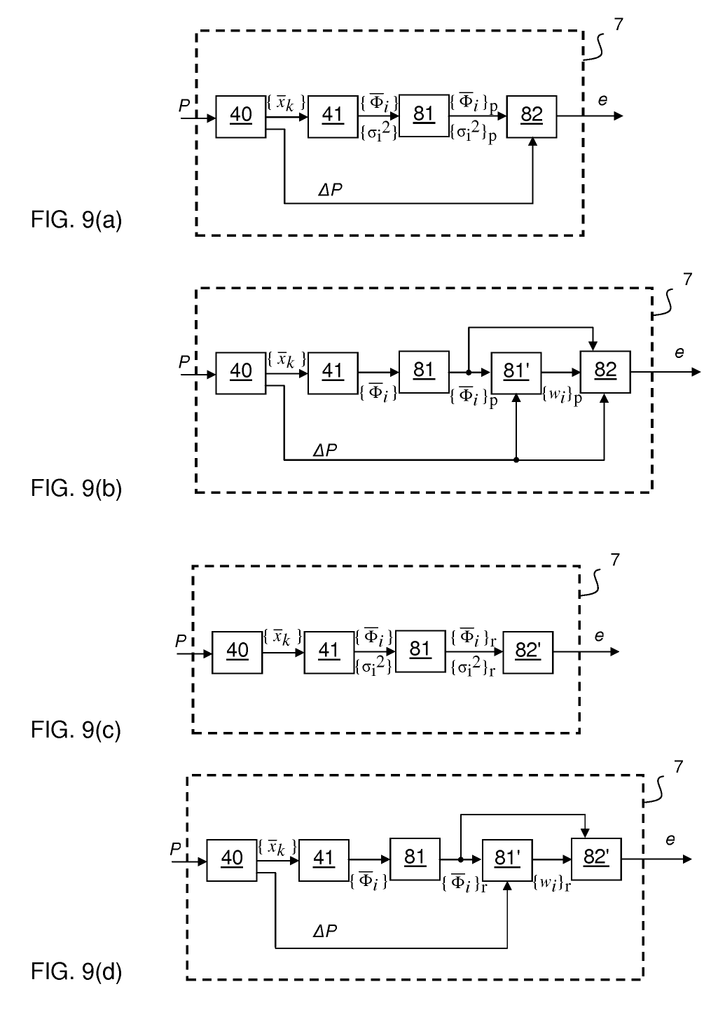

FIGS. 9(a)-9(d) are block diagrams of filtering devices 7 that operate according a respective implementation of the method of FIG. 5.

The device in FIG. 9(a) implements step 508 to subtract principal components according to the averaged implementation. The device 7 comprises the extraction module 40 and the PCA module 41 as described in relation to FIG. 4(b). The extraction module 40 implements steps 501-504 and provides the populated signal vectors x̅ₖ as well as the sequence of pressure values within the time window, indicated by ΔP. The PCA module 41 implements step 505, by operating on the signal vectors x̅ₖ to compute and provide a set of principal components {Ψ̅ᵢ} and a set of variances {σᵢ²}. A selection module 81 operates on the sets {Ψ̅ᵢ} and {σᵢ²} to select and provide the subsets {Ψ̅ᵢ}ₚ and {σᵢ²}ₚ representing the pump pulses within the time window ΔPₜ. A subtraction module 82 operates on the subsets {Ψ̅ᵢ}ₚ and {σᵢ²}ₚ and the pressure values ΔP to compute, adjust and subtract the averaged pump profile from the pressure values ΔP, and to generate and output the filtered signal e. It is realized that modules 81 and 82 correspond to the filtering module 42 in FIGS. 4(a)-4(b).

The device 7 in FIG. 9(b) implements step 508 to subtract principal components according to the fitted implementation. Compared to FIG. 9(a), the PCA module 41 only provides a set of principal components {Ψ̅ᵢ}, ordered by magnitude of the associated variances. As seen, the PCA module 41 need not provide the set of variances {σᵢ²}. The selection module 81 operates on the set {Ψ̅ᵢ} to select and provide the subset {Ψ̅ᵢ}ₚ. A correlation module 81′ operates on the subset {Ψ̅ᵢ}ₚ and the pressure values ΔP to generate the set of weights {wᵢ}ₚ. The subtraction module 82 operates on the subset {Ψ̅ᵢ}ₚ, the set of weights {wᵢ}ₚ and the pressure values ΔP to compute and subtract fitted pump profiles from the pressure values ΔP, and to generate and output the filtered signal e. Modules 81, 81′ and 82 correspond to the filtering module 42 in FIGS. 4(a)-4(b).

The device in FIG. 9(c) implements step 508 to combine residual principal components according to the averaged implementation. Compared to FIG. 9(a), the extraction module 40 only provides the populated signal vectors {x̅ₖ}. The selection module 81 operates on the sets {Ψ̅ᵢ} and {σᵢ²} from the PCA module 41 to select and provide the residual subsets {Ψ̅ᵢ}ᵣ and {σᵢ²}ᵣ. A combination module 82′ operates on the subsets {Ψ̅ᵢ}ᵣ and {σᵢ²}ᵣ to compute the averaged filtered signal segment for the time window, and to generate and output the filtered signal e. Modules 81 and 82′ correspond to the filtering module 42 in FIGS. 4(a)-4(b).

The device 7 in FIG. 9(d) implements step 508 to combine residual principal components according to the fitted implementation. Compared to FIG. 9(b), the selection module 81 operates on the set {Ψ̅ᵢ} to select and provide the residual subset {Ψ̅ᵢ}ᵣ. The correlation module 81′ operates on the subset {Ψ̅ᵢ}ᵣ and the pressure values ΔP to generate the set of weights {wᵢ}ᵣ. A combination module 82′ operates on the subset {Ψ̅ᵢ}ᵣ and the set of weights {wᵢ}ᵣ to compute fitted filtered signal segments for the time window, and to generate and output the filtered signal e. Modules 81, 81′ and 82′ correspond to the filtering module 42 in FIGS. 4(a)-4(b).

It is to be understood that the structuring of the device 7 into functional modules as shown in FIGS. 4 and 9 is provided for the purpose of illustration. To the extent that device operates on the incoming data by digital processing, using one or more digital data processors 8 in conjunction with electronic memory 9 (FIG. 1), all or some of the modules may not be physically present in the device 7 and the data is not conveyed between the modules, but rather written to and read from the electronic memory 9 by the one or more digital data processors 8.

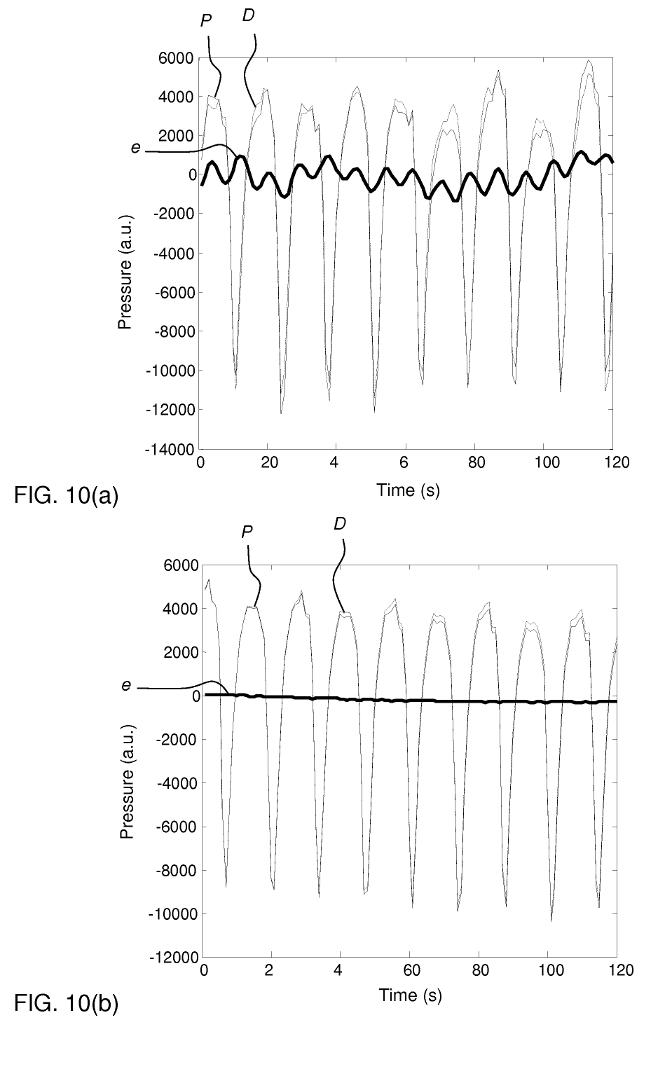

FIG. 10(a) illustrates (by a thin solid line) the pressure signal P acquired from the venous pressure sensor 6b in FIG. 1 while both of the access devices 2′, 2″ are connected to the vascular access 3 of the subject. A corresponding filtered signal e is illustrated by a thicker solid line. The filtered signal e is generated by the device 7 in FIG. 9(b), i.e. by subtracting fitted pump profiles from the pressure signal P. As seen, the filtered signal e includes periodic pulsations that originate from the heart in the subject. It can also be seen that the periodic pulsations are superimposed on a varying baseline, which may at least partly be attributed to the breathing of the patient. For the purpose of illustration, FIG. 10(a) also illustrates, by dashed lines, a difference signal D which is formed by subtracting the filtered signal e from the pressure signal P. The difference signal D thereby represents an aggregation of the fitted pump profiles that have been subtracted from the pressure signal P when generating the filtered signal e. FIG. 10(b) illustrates the pressure signal P acquired from the venous pressure sensor 6b in FIG. 1 when the access device 2″ on the venous side (“venous access device” or “venous needle”) has been dislodged from the vascular access 3. As seen, the heart pulsations have disappeared in the filtered signal e.

Reverting to step 506 in FIG. 5, the subset {Ψ̅ᵢ}ₚ of principal components may be selected based on a reference signal, instead of being identified by an initial principal component selected based on variance. The reference signal, which is received by the filtering device 7, may be generated by a reference sensor in the EC circuit 1 to indicate the current operating frequency of the pump 4. The reference signal thus indicates any one of the frequencies 0.5f₀, f₀, etc. Knowing one of these frequencies, step 506 may readily identify all principal components that primarily represent the pump pulses. In one implementation, step 506 may determine the dominating frequency of the principal components and compare the dominating frequencies to the current operating frequency. In another implementation, step 506 may generate harmonic vectors at the frequencies 0.5f₀, f₀, etc and cross-correlate the harmonic vectors with the principal components. The reference signal may be obtained from a tachometer associated with the pump 4 to measure the rotation speed of an element (e.g. the rotor 12) in the power transmission of the pump 4. Alternatively, the reference signal may be a control signal for the pump 4, or a pressure signal generated by a pressure sensor in the EC circuit 1. In fact, the pressure signal to be filtered may be used as the reference signal, provided that the current operating frequency can be reasonably well approximated from the pulsations in the pressure signal.

It should be understood that the method in FIG. 5 is equally applicable to processing of more than one pressure signal, e.g. as implemented by the device 7 shown in FIG. 4(a), although certain steps of the method in FIG. 5 may need to be slightly modified. Step 502 may be modified, if present, to pre-process all of the incoming pressure signals. Step 502 may include generating the above-mentioned intermediate signal by linearly combining two or more of the incoming pressure signals. Step 504 may be modified to populate the signal vectors so as to correspond to at least one signal segment in all of the incoming pressure signals. In one example, step 504 populates only one signal vector based on each pressure signal. In another example, step 504 populates at least a subset of the signal vectors to correspond to a plurality of time-shifted signal segments in two or more of the incoming pressure signals. In yet another example, step 504 populates at least a subset of the signal vectors to correspond to a plurality of time-shifted signal segments in the intermediate signal generated by step 502. It can be shown that computing principal components based on signal vectors given by time-shifted segments in such an intermediate signal is mathematically equivalent to computing principal components based on signal vectors given by time-shifted segments in the pressure signals that are linearly combined into the intermediate signal. Step 508 may be implemented to generate the filtered signal e by subtracting the subset {Ψ̅ᵢ}ₚ from any one of the incoming pressure signals, using the weights determined in step 507, e.g. according to the averaged implementation or the fitted implementation presented above. Alternatively, step 508 may be implemented to combine the subset {Ψ̅ᵢ}ᵣ into the filtered signal e, e.g. according to the averaged implementation or the fitted implementation presented above.

The skilled person is readily able to modify the devices in FIGS. 9(a)-9(d) for processing of more than one pressure signal in accordance with the foregoing.

It should also be noted that FIG. 1 is merely given as a simplified example of an EC circuit 1, which may include further pressure sensors, e.g. at least one pressure sensor between the blood pump 4 and the blood processing unit 5, and at least one pressure sensor in a circuit (not shown) for pumping a treatment fluid (e.g. a dialysis fluid) through the blood processing unit 5. The pressure signals from these pressure sensors may be supplied to and processed by the filtering device 7, in addition to or instead of the pressure signals from the sensors 6a, 6b in FIG. 1.

Irrespective of representation, the filtering device 7 may be implemented by special-purpose software (or firmware) run on one or more general-purpose or special-purpose computing devices. In this context, it is to be understood that an “element” or “means” of such a computing device refers to a conceptual equivalent of a method step; there is not always a one-to-one correspondence between elements/means and particular pieces of hardware or software routines. One piece of hardware sometimes comprises different means/elements. For example, a processing unit serves as one element/means when executing one instruction, but serves as another element/means when executing another instruction. In addition, one element/means may be implemented by one instruction in some cases, but by a plurality of instructions in some other cases. Such a software controlled computing device may include one or more processing units (cf. 8 in FIG. 1), e.g. a CPU (“Central Processing Unit”), a DSP (“Digital Signal Processor”), an ASIC (“Application-Specific Integrated Circuit”), discrete analog and/or digital components, or some other programmable logical device, such as an FPGA (“Field Programmable Gate Array”). The device 7 may further include a system memory and a system bus that couples various system components including the system memory (cf. 9 in FIG. 1) to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAM) and flash memory. The special-purpose software may be stored in the system memory, or on other removable/non-removable volatile/non-volatile computer storage media which is included in or accessible to the computing device, such as magnetic media, optical media, flash memory cards, digital tape, solid state RAM, solid state ROM, etc. The device 7 may include one or more communication interfaces, such as a serial interface, a parallel interface, a USB interface, a wireless interface, a network adapter, etc, as well as one or more data acquisition devices, such as an A/D converter. The special-purpose software may be provided to the device 7 on any suitable computer-readable medium, including a record medium or a read-only memory.

It is also conceivable that some (or all) elements/means are fully or partially implemented by dedicated hardware, such as an FPGA, an ASIC, or an assembly of discrete electronic components (resistors, capacitors, operational amplifier, transistors, filters, etc), as is well-known in the art.

It should be emphasized that the invention is not limited to digital signal processing, but could be fully implemented by a combination of analog devices.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

The inventive filtering technique is applicable for processing a pressure signal obtained from one or more pressure sensors in all types of fluid containing systems, especially in systems for medical or therapeutic use, to suppress or essentially remove interferences (“first pulses”) originating from a pulse generator, which is located in or is associated with the fluid containing system. In this context, “associated with” implies that the pulse generator need not be included in the fluid containing system but is capable of generating pressure waves that propagate in the fluid containing system to the pressure sensor(s). The resulting filtered signal contains pressure variations (“second pulses”). The inventive filtering technique allows the filtered signal to be processed for analysis of the pressure variations, for any purpose, irrespective of the interferences in the pressure signal.

For example, the inventive filtering technique is applicable in all types of EC circuits in which blood is taken from the systemic blood circuit of the patient to have a process applied to it before it is returned to the patient. Such EC circuits include circuits for hemodialysis, hemofiltration, hemodiafiltration, plasmapheresis, apheresis, extracorporeal membrane oxygenation, assisted blood circulation, and extracorporeal liver support/dialysis. The inventive technique is likewise applicable for filtering in other types of EC circuits, such as circuits for blood transfusion, as well as heart-lung-machines.

The inventive technique is also applicable to fluid systems that contain other liquids than blood and are connected to the cardiovascular system of a human or animal subject, including systems for intravenous therapy, infusion pumps, automated peritoneal dialysis (ΔPD) systems, etc. Examples of such liquids include medical solutions, dialysis fluids, infusion liquids, water, etc.

It should be emphasized that the fluid containing system need not be connected to a human or animal subject. For example, the fluid containing system may be a regeneration system for dialysis fluid, which circulates dialysis fluid from a supply through a regeneration device and back to the supply. In another example, the fluid containing system is an arrangement for priming an EC circuit by pumping a priming fluid from a supply via the EC circuit to a dialyser. In a further example, the fluid containing system is an arrangement for purifying water, which pumps water from a supply through a purifying device.

The inventive technique is applicable for removing or suppressing pressure pulses that originate from any type of pulse generator, be it mechanic or human, which is arranged in or associated with the fluid containing system. The pulse generator may be any type of pumping device, not only rotary peristaltic pumps as disclosed above, including other types of positive displacement pumps, such as linear peristaltic pumps, diaphragm pumps, as well as centrifugal pumps. Further, the pulse generator may be one or more valves or flow restrictors that are installed in or associated with the fluid containing system. The valves and flow restrictors may be operable to intermittently stop a flow of fluid, change a flow rate of fluid, or change a fluid flow path. The valves and flow restrictors may also be included in a system for degassing of a fluid or a system for changing the static pressure of a fluid. In another example, the pulse generator is a balancing chamber as used in certain types of dialysis systems.

Likewise, the inventive technique is applicable to produce a filtered signal with pressure variations or pressure pulses (“second pulses”) originating from any type of pulse generator, be it human or mechanic.

The inventive technique need not operate on real-time data, but could be used for processing off-line data, such as a previously recorded pressure signal.

1. A signal filtering device, comprising:
an input for receiving at least one pressure signal from a set of pressure sensors in a fluid containing system, the at least one pressure signal comprising first pulses and second pulses, the first pulses originating from a first pulse generator; and
a signal processor connected to the input and being configured to:
populate a plurality of signal vectors of identical length such that each of the signal vectors corresponds to a respective signal segment of signal values in the at least one pressure signal,
process the signal vectors by Principal Component Analysis, PCA, so as to compute principal components for the signal vectors,
select, among the principal components, either a first subset of principal components that primarily represent the first pulses or a second subset of principal components that are excluded from the first subset,
determine a first set of weights for the principal components in the first subset or a second set of weights for the principal components in the second subset, and
generate a filtered signal by: (i) subtracting, as a function of the first set of weights, the principal components in the first subset from the at least one pressure signal, or (ii) combining, as a function of the second set of weights, the principal components in the second subset.

2. The signal filtering device of claim 1, wherein the first pulse generator is periodic, and wherein the signal processor is configured to populate the signal vectors such that at least a subset of the signal vectors corresponds to mutually time-shifted signal segments within a time window in a dedicated pressure signal included among the at least one pressure signal, or in an intermediate signal generated based on the at least one pressure signal.

3. The signal filtering device of claim 2, wherein the signal processor is configured to populate the signal vectors such that said at least a subset of the signal vectors corresponds to partly overlapping and mutually time-shifted signal segments in the dedicated pressure signal or the intermediate signal.

4. The signal filtering device of claim 2, wherein the signal processor is configured to populate the signal vectors exclusively based on the mutually time-shifted signal segments in the dedicated pressure signal or the intermediate signal.

5. The signal filtering device of claim 1, wherein the at least one pressure signal comprises a plurality of pressure signals from a plurality of pressure sensors in the fluid containing system, and wherein the signal processor is configured to populate the signal vectors such that each signal vector corresponds to a respective signal segment in a respective one of the pressure signals.

6. The signal filtering device of claim 1, wherein the signal processor is configured, when selecting the first or second subset, to:
determine a current frequency associated with the first pulses;
identify, among the principal components computed by PCA, a group of principal components that have a dominating frequency with a predefined relation to the current frequency; and
select the first or second subset based on said group of principal components.

7. The signal filtering device of claim 6, wherein the signal processor is configured to select said group of principal components as the first subset.

8. The signal filtering device of claim 6, wherein the signal processor is configured to the select the second subset among the principal components computed by PCA while excluding said group of principal components.

9. The signal filtering device of claim 6, wherein the signal processor is configured, when processing the signal vectors by PCA, to compute variances associated with the principal components, and wherein the signal processor is configured, when determining the current frequency, to select an initial principal component among the principal components computed by PCA based on a magnitude of the variances associated with the principal components, such that the initial principal component represents the current frequency.

10. The signal filtering device of claim 9, wherein the first pulses exceed the second pulses in magnitude in the at least one pressure signal and signal vector values in each of the signal vectors are zero on average, and wherein the signal processor is configured to select, as the initial principal component, the principal component associated with the largest variance among the variances computed by PCA.

11. The signal filtering device of claim 9, wherein the signal processor is configured, when identifying said group of principal components, to identify all principal components that have a dominating frequency which is a multiple of the dominating frequency of the initial principal component.

12. The signal filtering device of claim 6, wherein the first pulse generator comprises a peristaltic pump with a number of rollers, and wherein the signal processor is configured, when identifying said group of principal components, to identify all principal components that have a dominating frequency which is a multiple of the dominating frequency of the initial principal component divided by the number of rollers.

13. The signal filtering device of claim 1, wherein the signal processor is configured, when generating the filtered signal, to subtract, from the at least one pressure signal, a linear combination of the principal components in the first subset, the first set of weights forming coefficients in the linear combination, or linearly combine the principal components in the second subset using the second set of weights as coefficients.

14. The signal filtering device of claim 13, wherein the signal processor is configured, when processing the signal vectors by PCA, to compute variances associated with the principal components, and wherein the signal processor is configured to determine the first or second set of weights as a function of the variances associated with the principal components in the first or second subset, respectively.

15. The signal filtering device of claim 13, wherein the signal processor is configured, when determining the first or second set of weights, to compute, for each one of a plurality of signal segments in the at least one pressure signal, a respective set of correlation values estimating correlations between each of the principal components in the first or second subset and the signal vector corresponding to said each one of the plurality of signal segments, and determine the first or second set of weights as a function of the respective set of correlation values; and wherein the signal processor is configured, when generating the filtered signal, to generate, as a function of the first or second set of weights determined for said each one of the plurality of signal segments, a filtered signal segment, so as to produce a plurality of filtered signal segments for the plurality of signal segments.

16. The signal filtering device of claim 15, wherein the signal processor is configured to generate the filtered signal by concatenating at least parts of the filtered signal segments.

17. The signal filtering device of claim 15, wherein the signal processor is configured to generate the filtered signal by combining overlapping parts of the filtered signal segments.

18. The signal filtering device of claim 1, wherein the signal processor is further configured, when processing the signal vectors by PCA, to process the signal vectors to compute eigenvectors for an estimated covariance matrix comprising estimated covariance values for the signal vectors.

19. The signal filtering device of claim 18, wherein the estimated covariance matrix is given by ƒ(X^{T}X), wherein X is a matrix with the signal vectors arranged as rows or columns, X^{T} is a transpose of the matrix X, and ƒ is a linear function.

20. The signal filtering device of claim 18, wherein the signal processor is further configured, when processing the signal vectors by PCA, to compute the estimated covariance values, populate the estimated covariance matrix by the estimated covariance values, and process the estimated covariance matrix for computation of the eigenvectors.

21. The signal filtering device of claim 18, wherein the signal processor is further configured, when processing the signal vectors by PCA, to process the eigenvectors to generate a set of mutually independent eigenvectors for the estimated covariance matrix, and obtain the principal components based on the set of mutually independent eigenvectors.

22. The signal filtering device of claim 1, wherein the signal processor is further configured, before processing the signal vectors by PCA, to process the at least one pressure signal and/or the signal vectors to yield an average of zero for signal vector values in the respective signal vector.

23. The signal filtering device of claim 1, wherein the fluid containing system comprises an extracorporeal blood circuit fluidly connected to a vascular system in a human body, wherein the first pulse generator comprises a pumping device in the extracorporeal blood circuit, and wherein the second pulses originate from a second pulse generator in or attached to the human body.

24. A signal filtering device, comprising:
means for receiving at least one pressure signal originating from a set of pressure sensors in a fluid containing system, the at least one pressure signal comprising first pulses and second pulses, the first pulses originating from a first pulse generator;
means for populating a plurality of signal vectors of identical length such that each of the signal vectors corresponds to a respective signal segment of signal values in the at least one pressure signal;
means for processing the signal vectors by Principal Component Analysis, PCA, so as to compute principal components for the signal vectors;
means for selecting, among the principal components, either a first subset of principal components that primarily represent the first pulses or a second subset of principal components that are excluded from the first subset;
means for determining a first set of weights for the principal components in the first subset or a second set of weights for the principal components in the second subset; and
means for generating a filtered signal by: (i) subtracting, as a function of the first set of weights, the principal components in the first subset from the at least one pressure signal, or (ii) combining, as a function of the second set of weights, the principal components in the second subset.

25. A filtering method performed by a data processor and comprising:
receiving at least one pressure signal originating from a set of pressure sensors in a fluid containing system, the at least one pressure signal comprising first pulses and second pulses, the first pulses originating from a first pulse generator;
populating a plurality of signal vectors of identical length such that each of the signal vectors corresponds to a respective signal segment of signal values in the at least one pressure signal;
processing the signal vectors by Principal Component Analysis, PCA, so as to compute principal components for the signal vectors;
selecting, among the principal components, either a first subset of principal components that primarily represent the first pulses or a second subset of principal components that are excluded from the first subset;
determining a first set of weights for the principal components in the first subset or a second set of weights for the principal components in the second subset; and
generating a filtered signal by: (i) subtracting, as a function of the first set of weights, the principal components in the first subset from the at least one pressure signal, or (ii) combining, as a function of the second set of weights, the principal components in the second subset.

26. A computer-readable medium comprising processing instructions for causing the data processor to perform the method of claim 25.
